(19) United States
(12) Patent Application Publication
(10) Pub. No.: US 2005/0055167 A1
(43) Pub. Date: Mar. 10, 2005

(54) DNA based number system and arithmetic

(75) Inventors: Lalit M. Bharadwaj, Chandigarh (IN); Awdhesh Kumar Shukla, Chandigarh (IN); Amol P. Bhondekar, Chandigarh (IN); Rakesh Kumar, Chandigarh (IN); Ram Prakash Bajpai, Chandigarh (IN)

Correspondence Address: LADAS & PARRY, 26 WEST 61ST STREET, NEW YORK, NY, 10023, US

(73) Assignee: COUNCIL OF SCIENTIFIC AND INDUSTRIAL RESEARCH

(21) Appl. No.: 10/849,491

(22) Filed: May 19, 2004

Related U.S. Application Data
(60) Provisional application No. 60/472,001, filed on May 20, 2003.

(51) Int. Cl. G06F 19/00; G01N 33/48; G01N 33/50

(52) U.S. Cl. 702/20

(57) ABSTRACT

A DNA based number system and basic arithmetic operations i.e. addition and subtraction for this have been developed. The method of invention comprises assignment of arbitrary integer values to all DNA bases (A=0, T=1, C=2, G=3), arbitrary assignment of complements of DNA bases (A=G, complement of T=C and vice-versa), representation of integers and real numbers in terms of DNA bases and performing basic arithmetic assignment on DNA based number system. This will find wide application in the area of DNA or other molecular computation devices and processors.

### FIELD OF THE INVENTION

The present invention relates to a DNA based number system and arithmetic. More particularly, the present invention relates to a DNA based number system and arithmetic which comprises assignment of arbitrary values to all DNA bases, arbitrary assignment of complements of DNA bases, representation of integers and real numbers in terms of DNA bases and performing basic arithmetic assignment on DNA based number system.

### BACKGROUND TO THE INVENTION

Nature has perfected the technique for creation of diverse living species with wide ranging shapes, sizes and characteristics over a period spanning millions of years. Deoxyribose Nucleic Acid (DNA), the carrier of genetic information can be considered to be a powerful and complex molecular electronic device. The question is how this wonderful molecular device. DNA can be utilized to fabricate electronic components. DNA has the power to store and retain the genetic information, which can be retrieved as and when required for execution of biological processes and for growth and maintenance of all kind of living organisms from smallest microbes to giant whales. All living organisms are formed by differentiation of single cell called zygote, formed during reproduction. This cell does not contain any component of body i.e. zygote does not contain bones or teeth but DNA of zygote has all the protocols for the formation of all the organs of a living system. This is all possible only when DNA could communicate information throughout its length by charge transport.

Today's computer can be built out of any bi-stable device which means that the elements of the computer must have two stable positions or states. These two stable states are ON and OFF and represent 1 and 0 respectively. The basic building block of most computers today is the transistor which is made up of semiconductor. The change to ON or OFF is done electrically, so the speed is quite fast. A transistor can change states in roughly 3 billionths of a second and about 10 million transistors can be fitted into a space 1 cm² area of Silicon or Gallium Arsenide based Integrated Circuit.

A tremendous amount of research is being done to devise methods for DNA based computing and molecular electronic alternatives since semiconductor devices are approaching limits in terms of speed and miniaturization. DNA is considered a promising material for use in the design and fabrication of high density memory devices and ultra high speed electronic devices. Interest in the study of charge transport in DNA has grown during last few years since DNA can be used as nano-component: as an insulator, semiconductor and conductor/proximity induced superconductor [1,2,3,4] depending upon the base sequence, length and orientation. DNA can be coated selectively on metals with molecular level precision [1,5] thereby providing capability to design molecular electronic components such as diode, triode, transistor, etc. DNA can also handle massive parallel processing [6,7,8], is extremely energy efficient, size and characteristics are controllable, have a tremendous ability to store information, are readily available; synthesis of any imaginable sequence; and are environmental friendly. In addition DNA possesses four bases (AGTC) instead of 0 and 1.

It is important to devise computers based on quad-state device made up of DNA. These four states would be represented by 0, 1, 2 and 3. These states would either be distinguished by measuring current levels or by measuring optical difference. When four DNA bases (ATGC) are put together and considered as a single unit for DNA based computing, about 3×10¹³ such units can be placed within 1 cm² area. In order to work upon DNA based devices, it is however essential to provide a suitable DNA based number system to perform DNA based arithmetic. A DNA based number system and software to translate this system has therefore been developed and is disclosed herein, which also enables the creation of DNA based quad-state devices.

### OBJECTS OF THE INVENTION

The main object of the present invention is to develop DNA based number system and perform DNA based arithmetic and also software for encoding of DNA based numbers and performs arithmetic operations over DNA encoded numbers.

Another object of the present invention is to define encoding of both positive and negative integers in terms of DNA bases.

Yet another object of the present invention is to define encoding of both positive and negative real numbers in terms of DNA bases

Yet another object of the present invention is to define basic arithmetic operations on DNA encoded integers and real numbers.

### SUMMARY OF THE INVENTION

The present invention relates to a DNA based number system and arithmetic. More particularly, the present invention relates to a DNA based number system and arithmetic which comprises assignment of arbitrary values to all DNA bases, arbitrary assignment of complements of DNA bases, representation of integers and real numbers in terms of DNA bases and performing basic arithmetic assignment on DNA based number system.

In an embodiment of the present invention there are 4 elements in DNA based number system. These elements are “A”, “T”, “C”, “G”.

In an another embodiment of the present invention the arbitrary values assigned to each element of the DNA based number system are: A=0, T=1, C=2, G=3.

In an embodiment of the present invention the integers are represented as 8 bases/cell. Complement representation is used to represent integers. Positive integers don't have complements.

In an another embodiment of the present invention the assigned complements to the elements of DNA based number system are: Complement of A=G, Complement of T=C and vice-versa.

In yet another embodiment of the present invention the value of base in the DNA based number system is positional.

In yet another embodiment of the present invention the conversion of positive integers to DNA based number is done by dividing the number by 4 and extracting the remainder, continuing this procedure until the quotient becomes zero. The first remainder digit extracted as the Least Significant Base (LSB), the last extracted digit will be marked as Main Significant Base (MSB), by writing the bases extracted left to right from MSD to LSD gives DNA based number, completing the cell (8-bases/cell or its multiple) by adding extra padding to the left as As and putting leftmost base as sign base i.e. “A” for positive integer. Leftmost base would represent the sign of the number.

In yet another embodiment of the present invention the conversion of negative numbers to DNA based numbers is done by, taking the number as positive integer, converting it to DNA based number system, producing its complement by changing As to Gs, Ts to Cs and vice versa, adding a base T (=1) to the complement, completing the cell (8-bases/cell or its multiple) by adding extra padding to the left as Gs and putting leftmost base as sign base i.e. “G” for negative integer. Leftmost base would represent the sign of the number.

In yet another embodiment of the present invention the real numbers are represented as floating-point representation in 32-bases.

In yet another embodiment of the present invention for the conversion of positive real numbers to DNA based number the real number is first converted to integer by right shifting the point of decimal, this integer is then converted to DNA based number as in the case positive integer to DNA based number conversion. The number of points shifted is recorded and represented as exponent (utilizing integer to DNA based conversion scheme as mentioned above), leftmost base represents sign base of the number, next 23-bases represent the magnitude and the rest 8-bases represent the exponent.

In yet another embodiment of the present invention the sign base in the case of positive real number would be “T” and sign base in the case of negative real number would be “C”.

In yet another embodiment of the present invention for the conversion of negative real numbers to DNA based number the real number is taken as positive real number, real number is then converted to integer by right shifting the point of decimal, this integer is then converted to DNA based number as in the case positive integer to DNA based number conversion, then complement of resulting DNA based number is produced by changing As to Gs, Ts to Cs and vice versa, adding a base T (=1) to the complement. The number of points decimal is shifted is recorded and represented as exponent (utilizing integer to DNA based conversion scheme as mentioned above), leftmost base represents sign base of the number, next 23-bases represent the magnitude and the rest 8-bases represent the exponent.

### BRIEF DESCRIPTION OF THE ACCOMPANYING DRAWINGS

FIG. 1. Process sheet for integer representation in terms of DNA bases and arithmetic

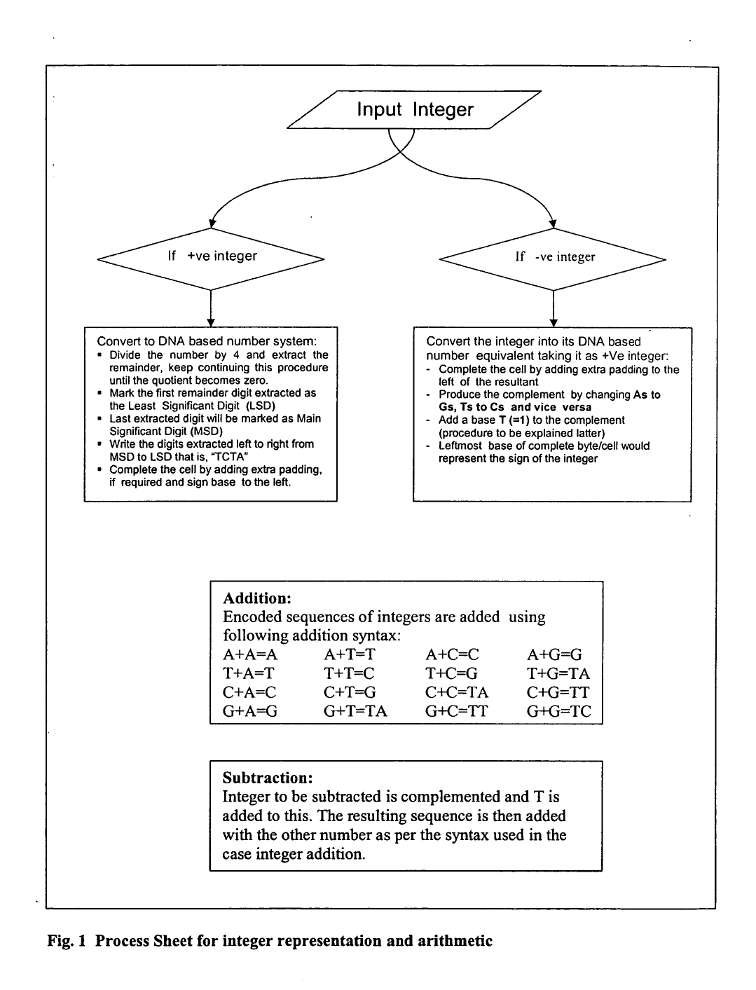

FIG. 2. Process sheet for real number representation in terms of DNA bases and arithmetic

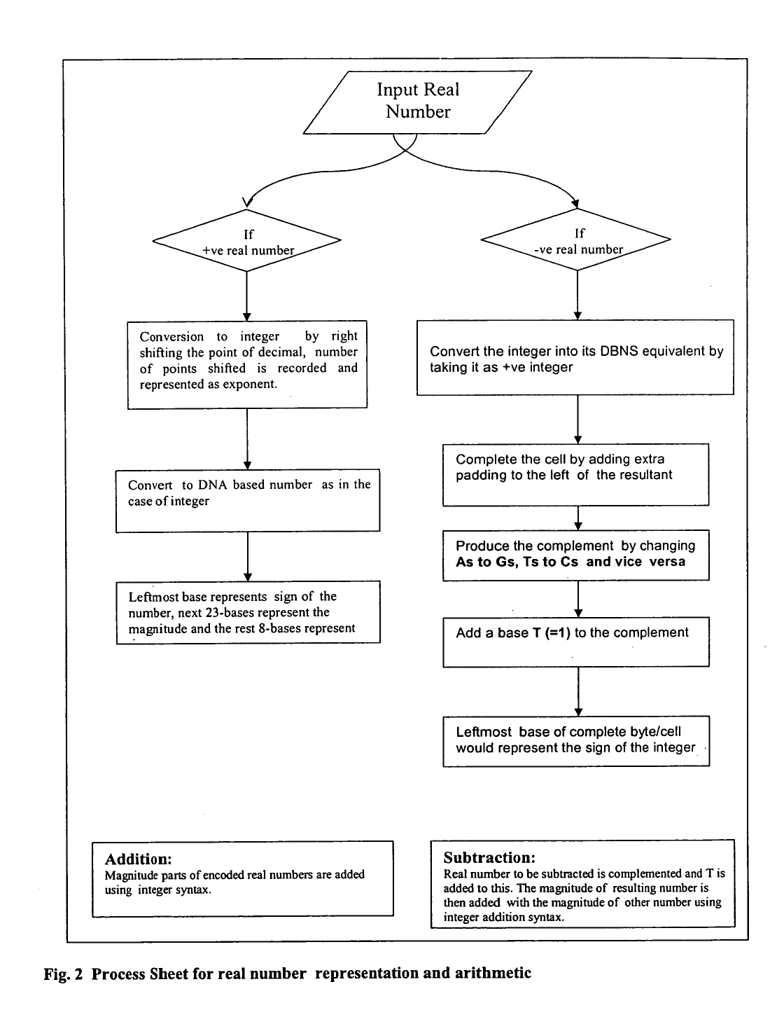

### DETAILED DESCRIPTION OF THE INVENTION

The present invention relates to a DNA based number system and arithmetic. More particularly, the present invention relates to a DNA based number system and arithmetic which comprises assignment of arbitrary values to all DNA bases, arbitrary assignment of complements of DNA bases, representation of integers and real numbers in terms of DNA bases and performing basic arithmetic assignment on DNA based number system.

In the system of the invention there are 4 elements in DNA based number system. These elements are “A”, “T”, “C”, “G”. Arbitrary values assigned to each element of the DNA based number system are: A=0, T=1, C=2, G=3. Integers can be represented in the form of 8 bases/cell. The value of base in the DNA based number system is positional.

Complement representation is used to represent integers. However, positive integers don't have complements. The assigned complements to the elements of DNA based number system are: Complement of A=G, Complement of T=C and vice-versa.

Conversion of positive integers to DNA based number is done by dividing the number by 4 and extracting the remainder, continuing this procedure until the quotient becomes zero. The first remainder digit extracted as the Least Significant Base (LSB), the last extracted digit will be marked as Main Significant Base (MSB). The DNA based number is obtained by writing the bases extracted left to right from MSD to LSD. The cell is then completed (8-bases/cell or its multiple) by adding extra padding to the left as A's and putting leftmost base as sign base i.e. “A” for positive integer. Leftmost base would represent the sign of the number.

The conversion of negative numbers to DNA based numbers is done by first taking the number as positive integer, converting it to DNA based number system as discussed above, producing its complement by changing As to Gs, Ts to Cs and vice versa, adding a base T (=1) to the complement, completing the cell (8-bases/cell or its multiple) by adding extra padding to the left as G's and putting leftmost base as sign base i.e. “G” for negative integer. Leftmost base would represent the sign of the number.

Real numbers are represented as floating-point representation in 32-bases.

Positive real numbers are converted to DNA based number by first converting the real number to an integer by shifting the decimal point to the right. This integer is then converted to DNA based number as discussed above with respect to positive integers. The number of points shifted is recorded and represented as an exponent (utilizing integer to DNA based conversion scheme mentioned above). The leftmost base represents sign base of the number, next 23-bases represent the magnitude and the rest 8-bases represent the exponent.

In the method of the invention, the sign base in the case of positive real number is be “T” and sign base in the case of negative real number is “C”.

For the conversion of negative real numbers to DNA based number the real number is first taken as positive real number and this real number then converted to an integer by shifting the decimal point to the right. This integer is then converted to DNA based number as in the case of positive integer to DNA based number conversion discussed above. The complement of resulting DNA based number is produced by changing A's to G's, T's to C's and vice versa and a base T (=1) is then added to the complement. The number of decimal points shifted is recorded and represented as exponent (utilizing integer to DNA based conversion scheme mentioned above). The leftmost base represents sign base of the number, next 23-bases represent the magnitude and the rest 8-bases represent the exponent.

The number system of the invention is useful in the creation of quad-state computing devices since the system has four elements and is not limited to the two elements of the binary system. This therefore enables representation of larger numbers when compared to the conventional binary system and therefore enables design and fabrication of powerful DNA based computing devices.

The invention will now be described with reference to the accompanying examples which are illustrative and should not be construed as limiting the scope of the invention in any manner.

### EXAMPLES

| S. No. | Description | DNA Based Number |
|---|---|---|
| 1. | Decimal number to | Integers are represented as 8 bases/cell |
| | DNA based number | Leftmost base represents the sign of the integer |
| | conversion | Decimal number to DNA based number conversion |
| | | 4 |
| | | 100 |
| | | Remainder |
| | | 4 |
| | | 25 |
| | | 0 = A LSD |
| | | 4 |
| | | 6 |
| | | 1 = T |
| | | 4 |
| | | 1 |
| | | 2 = C |
| | | 0 |
| | | 1 = T MSD |
| 2. | Limits to integer | Maximum: +4ⁿ⁻¹ − 1 |
| | representation in n | Minimum: −4ⁿ⁻¹ |
| | bases/cell | |
| 3. | Integer addition | Addition of 100 and 63: |
| | | 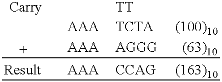 |
| 4. | Integer subtraction | Subtracting 63 from 100: |
| | | Sol.Complement of (63)₁₀ is taken and added to (100)₁₀ |
| | | 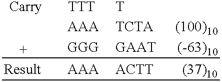 |
| | | Note: Extra carry T has to be ignored |
| 5. | Real number | Real numbers are represented as Floating-Point in 32-bases/cell. |
| | representation | Having three components i.e. sign bit, magnitude and exponent: |
| | | − leftmost base represents the sign |
| | | + next 23 bases represent the magnitude |
| | | + rest 8 bases represent exponent |
| | | − Sign base “T” represents positive real number |
| | | − Sign base “C” represents negative real number |
| | | 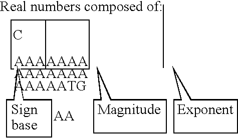 |
| 6. | Real number | Addition of 1.1 and 1.1 |
| | addition | Soln. Magnitude is taken for prcocessing: |
| | | 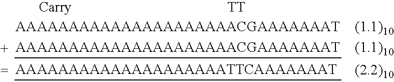 |
| 7. | Real number | Subtracting 12.3 from 10.1 |
| | subtraction | Soln. Addition of 10.1 and −12.3 would give the result |
| 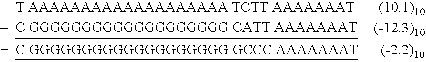 | | |

References
1. Braun, E., Eichen, Y., Sivan, U. & Ben-Yoseph, G. DNA-templated assembly and electrode attachment of a conducting silver wire. Nature. 391, 775-778 (1998).
2. Kasumov, A. Y., Kociak, M., Gueron, B., Reulet, B., Volkov, V. T., Klinov, D. V & Bouchiat, H. Proximity-induced superconductivity in DNA. Science. 291, 280-282 (2001).
3. Porath, D., Bezryadin, A., De Vries, S. & Dekker, C. Direct measurement of electrical transport through DNA molecules. Nature. 403, 635-638 (2000).
4. Fink, H. W. & Schonenberger, C. Electrical Conduction through DNA Molecules. Nature. 398,407410 (1999).
5. Winfree, E., Liu, F., Wenzler, L. A. and Seeman, N. C. Design and self-assembly of two-dimensional DNA crystals. Nature. 394, 539-544 (1998).
6. Adleman, L. M. Computing with DNA. Sci. Am. 54-61 (August 1998)
7. Adleman, L. M. Molecular computation of solutions to combinatorial problems. Science. 266, 1021-1024 (1994)
8. Benenson, Y., Elizur, T. P., Adar, R., Keinan, E., Livneh, Z. and Shapiro, E. Programmable and autonomous computing machines made of biomolecules. Nature. 414, 430-434 (2001)

1. A DNA based number system wherein the system has four bases comprising A, T, C and G and wherein each base is assigned an arbitrary value comprising A=0, T=1, C=2, G=3 and wherein both integers and real numbers are represented in the form of DNA bases, the value of a the base in the system being positional.

2. A system as claimed in claim 1, wherein real numbers are represented as floating-point representation in 32-bases.

3. A method for representing numbers in the form of DNA bases (A, T, C, G) comprising:
a) assigning arbitrary values to each DNA base wherein A=0, T=1, C=2, G=3;
b) assigning arbitrary complementary values to each DNA base such that complement of A=G, complement of T=C and vice-versa.

4. A method as claimed in claim 3, wherein the number is selected from the group consisting of a positive integer, a negative integer, a positive real number and a negative real number.

5. A method as claimed in claim 3, wherein the assigned complements to the elements of DNA based number system are: Complement of A=G, Complement of T=C and vice-versa.

6. A method as claimed in claim 3, wherein the value of base in the DNA based number system is positional.

7. A method as claimed in claim 4, wherein the positive integer is converted into the DNA base representation by:
(a) dividing the positive integer so obtained by four and extracting the remainder;
(b) repeating step (a) till a quotient of 0 is reached;
(c) marking the first remainder digit as the lest significant digit (LSD);
(d) marking the last extracted digit as the most main significant digit (MSD);
(e) writing the digits extracted from left to right from MSD to LSD; and
(f) completing a cell by adding padding if required, and a sign base to the left.

8. A method as claimed in claim 4, wherein the negative integer is converted to a DNA base representation thereof by;
(a) first changing the negative integer into a positive integer;
(b) dividing the positive integer so obtained by four and extracting the remainder;
(c) repeating step (c) till a quotient of 0 is reached;
(d) marking the first remainder digit as the lest significant digit (LSD);
(e) marking the last extracted digit as the most main significant digit (MSD);
(f) writing the digits extracted from left to right from MSD to LSD; and
(g) completing a cell by adding padding if required, and a sign base to the left;
(h) producing a complement by changing the A's to G's and T's to C's and vice versa;
(i) adding a base T (=1) to the complement
wherein the left most base of the completed byte/cell represents the sign of the integer.

9. A method as claimed in claim 4, wherein the positive real number is converted into a DNA base representation thereof, comprising:
(a) first converting the positive real number into a positive integer by shifting the decimal point to the right
(b) dividing the positive integer so obtained by four and extracting the remainder;
(c) repeating step (b) till a quotient of 0 is reached;
(d) marking the first remainder digit as the lest significant digit (LSD);
(e) marking the last extracted digit as the most main significant digit (MSD);
(f) writing the digits extracted from left to right from MSD to LSD; and
(g) completing a cell by adding padding if required, and a sign base to the left.
(h) recording the number of points shifted and represented as an exponent
wherein the leftmost base represents sign base of the number, and next 23-bases represent the magnitude and the rest 8-bases represent the exponent.

10. A method as claimed in claim 4, wherein the sign base in the case of positive real number is “T” and sign base in the case of negative real number is “C”.

11. A method as claimed in claim 4, wherein a negative real number is converted into a DNA base representation thereof, the method comprising
(a) taking the negative real number as a positive real number;
(b) converting the positive real number into a positive integer by shifting the decimal point to the right
(c) dividing the positive integer so obtained by four and extracting the remainder;
(d) repeating step (b) till a quotient of 0 is reached;
(e) marking the first remainder digit as the lest significant digit (LSD);
(f) marking the last extracted digit as the most main significant digit (MSD);
(g) writing the digits extracted from left to right from MSD to LSD; and
(h) completing a cell by adding padding if required, and a sign base to the left.
(i) recording the number of points shifted and represented as an exponent
wherein the leftmost base represents sign base of the number, and next 23-bases represent the magnitude and the rest 8-bases represent the exponent.

12. A software based on the DNA based number system of claim 1 wherein:
a) integers are represented as 8 bases/cell and a complement representation is used to represent integers and wherein positive integers do not have complements and the leftmost base represents the sign of the integer;
b) and wherein real numbers are represented as 32 bases/cell using floating-point representation scheme, wherein the leftmost base represents the sign of the number, next 23 bases represent the magnitude of the number and rest 8 bases represent the exponent i.e. number of bases the decimal was shifted towards right to convert the real number to integer.


SEQUENCE LISTING (5 sequences)

SEQ ID NO: 1 - DNA, 31 bp, Artificial
  DNA bases used to represent integers in DNA based number system
aaaaaaaaaaaaaaaaaaaaacgaaaaaaat

SEQ ID NO: 2 - DNA, 8 bp, Artificial
  DNA bases used to represent integers in DNA based number system
aaaaaaat

SEQ ID NO: 3 - DNA, 32 bp, Artificial
  DNA bases used to represent integers in DNA based number system
taaaaaaaaaaaaaaaaaaatcttaaaaaaat

SEQ ID NO: 4 - DNA, 32 bp, Artificial
  DNA bases used to represent integers in DNA based number system
cgggggggggggggggggggcattaaaaaaat

SEQ ID NO: 5 - DNA, 32 bp, Artificial
  DNA bases used to represent integers in DNA based number system
cggggggggggggggggggggcccaaaaaaat